(19) United States
(12) Patent Application Publication
(10) Pub. No.: US 2019/0169916 A1
(43) Pub. Date: Jun. 6, 2019

(54) Carbon Defense

(71) Applicants: Tyler W. Morgan, Dallas, TX (US); Ryan Morgan, Burleson, TX (US)

(72) Inventors: Tyler W. Morgan, Dallas, TX (US); Ryan Morgan, Burleson, TX (US)

(21) Appl. No.: 16/207,368

(22) Filed: Dec. 3, 2018

Related U.S. Application Data
(60) Provisional application No. 62/593,789, filed on Dec. 1, 2017.

(51) Int. Cl. E05F 15/72; E05F 15/77; G08B 21/14; G01N 33/00

(52) U.S. Cl. CPC E05F 15/72; E05F 15/77; G08B 21/14; G01N 33/004; E05Y 2400/52; E05Y 2400/44; E05Y 2400/81; E05Y 2400/664; E05Y 2900/106

(57) ABSTRACT

The present invention relates to a home safety device for use with a garage area in the home to detect the level of carbon monoxide and alert the user or occupants in the home accordingly. The device is configured to continual monitoring of the carbon monoxide levels in the garage. When the carbon monoxide level reaches a dangerous level for a prescribed amount of time, the device sounds a high-decibel alarm and sends a signal to the electric garage door opener to open the garage door simultaneously. In response to the transmitted signal, the garage door opens to significantly increase the ventilation and lower the CO level within the garage. The fresh air disperses and dilutes the carbon dioxide rendering a much safer environment. The device further configured to alert the user via a text message.

### BACKGROUND OF THE INVENTION

### A. Technical field

The present invention generally relates to a home safety device. More specifically, the present invention relates to safety monitoring and alerting device for use with a garage area in the home to detect the level of carbon monoxide and alert the user or occupants in the home accordingly.

### B. Description of related art

Generally, carbon monoxide (CO) is a color and odorless gas produced by the burning of carbon based fuels, such as gasoline or natural gas. There are other sources of carbon monoxide, however, most likely encounter by humans is through the use of carbon based fuels. The adverse effect of the gas is combining with hemoglobin to form carboxyhemoglobin in the blood. This condition prevents the blood from carrying oxygen to the cells. The inhalation of carbon monoxide results in loss of consciousness, arrhythmias, seizures and eventually death. The average for dangerous level of carbon monoxide exposure is at 100 parts per million for the human body.

Lower levels of carbon monoxide poisoning causes persistent headaches, lightheadedness, depression, memory loss, confusion, nausea, and vomiting. Long-term exposures to low levels of carbon monoxide causes permanent health problems and may lead to early death in some cases. Higher levels of carbon monoxide poisoning were almost always fatal unless the situation rectified in time. Initial treatment for carbon monoxide poisoning is to remove the affected person from exposure immediately. Cardiopulmonary resuscitation (CPR) may be immediately required for those who have lost consciousness. Further, the administration of oxygen accelerates the separation of carbon monoxide from the carboxyhemoglobin to provide hemoglobin. The process for returning back to hemoglobin takes time, as the time is an important factor when exposure to carbon monoxide. Failure to administer oxygen or exposing the affected persons to fresh air in time often results in fatal outcomes or permanent health issues.

The internal combustion engine should never be left running in the garage. The carbon monoxide levels can build up so quickly that a person may lose consciousness before they realize there is a problem. A garage that is attached to the house is especially dangerous when an automobile is left running in the garage. The CO leaks into the house eventually and even well-sealed homes and buildings are susceptible. It is only a matter of time before the CO levels become dangerous for any enclosed space like a garage if the source is not removed. Unfortunately, the known lethality of leaving a running car in the closed garage has been used by persons to take their own lives. Identification of carbon monoxide levels and prevention of carbon monoxide poisoning is always the best route to resolve the above-mentioned issues. Safe operation of appliances, heaters, fireplaces, and internal combustion engines is a fundamental public health issue. Henceforth, carbon monoxide detectors are important in the home, like smoke alarms.

Currently, most garages are not monitored for carbon monoxide levels. This is primarily due to cost and because most often the audible alarm cannot be heard within the dwelling area. Above certain levels, it kills people and pets. The initial onset of CO poisoning is also very hard to recognize because the initial symptoms are flu-like. There are many sources of poisoning that are located in the garage, such as internal combustion engines on lawn equipment, gas powered generators, vehicles inadvertently, or on purpose, left on, gas space heaters, gas appliances, gas water heaters, etc. CO levels increases rapidly if the garage doors are closed, and if not detected, causes serious illness, or death.

Therefore, there is a need for a home safety device that detects and alerts the users when the air quality in the area reaches dangerous level or certain threshold level of carbon monoxide. Further, there is need for a device that provides a solution to lessen the amount of carbon monoxide level in a particular environment, such as garage.

### SUMMARY OF THE INVENTION

The present invention relates to a home safety device, which monitors the levels of gases such as, carbon monoxide (CO) in an environment, for example, garage area. In one embodiment, the device is an innovative and unique solution to carbon monoxide buildup in a closed garage. The device is conveniently mounted in the garage on a wall. The device is configured to continual monitoring of the carbon monoxide levels in the garage. When the carbon monoxide level reaches a dangerous level for a prescribed amount of time, the device sounds a high-decibel alarm and sends a signal to the electric garage door opener to open the garage door simultaneously. The large size of the garage door opening significantly increase the amount of fresh air into the garage instantly. The fresh air disperses and dilutes the carbon dioxide rendering a much safer environment. The high-decibel alarm alerts anyone in the vicinity to check the source of the CO gas and take proper action. In another embodiment, the present invention provides an option to shut down the CO producing appliances remotely.

In another embodiment, the device also alerts the user via a text message, when the device is activated, so that the users or occupants may take appropriate action. If no action is taken within the arranged amount of time, emergency services are contacted and provide information about the location and incident details.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1A shows a perspective view of a home safety device implemented in a building, in an embodiment of the present invention.

FIG. 1B shows a perspective view of the home safety device implemented in a building controlling the garage door opening, in an embodiment of the present invention.

FIG. 2 shows a perspective view of the home safety device in an embodiment of the present invention.

FIG. 3A shows a front view of the home safety device in an embodiment of the present invention.

FIG. 3B shows a top view of the home safety device in an embodiment of the present invention.

FIG. 3C shows a side view of the home safety device in an embodiment of the present invention.

FIG. 3D shows a keyhole slots in a back panel of the home safety device in an embodiment of the present invention.

### DETAILED DESCRIPTION OF EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive.

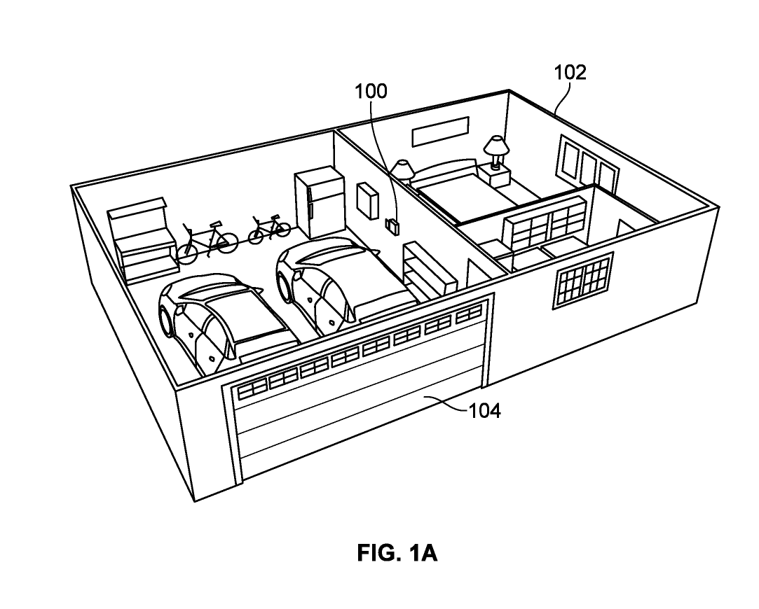

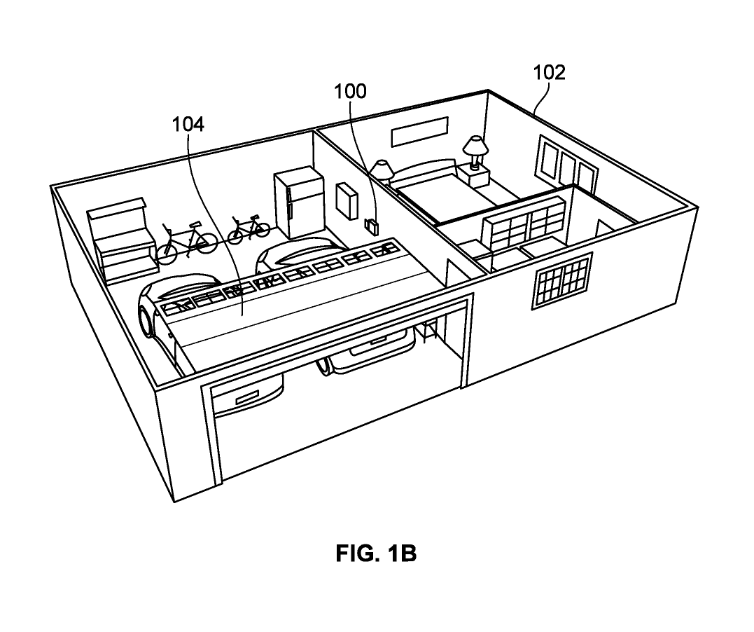

Referring to FIG. 1A, a home safety device 100, which monitors the levels of gases such as, carbon monoxide (CO) in an environment 102, for example, garage area is disclosed. The device 100 is configured to continuously monitor the level of carbon monoxide in the environment 102. The device 100 comprises a carbon monoxide detection module to monitor the level of carbon monoxide. When the levels of carbon monoxide arrive at a critical point, the device 100 is configured to activate and sounds a high-decibel alarm. Further, the activation simultaneously prompts to transmit a signal to a garage door opener to open the garage door 104, as shown in FIG. 1B. The opening of the garage door 104 allows fresh air into the garage area and disperses the carbon monoxide gas. Thereby, the device 100 prevents build-up of carbon monoxide and provides a safe environment. In another embodiment, the present invention provides an option to shut down the CO producing appliances remotely. This option allows to stop the operation of the CO producing appliance, when the level of carbon monoxide reaches a critical point.

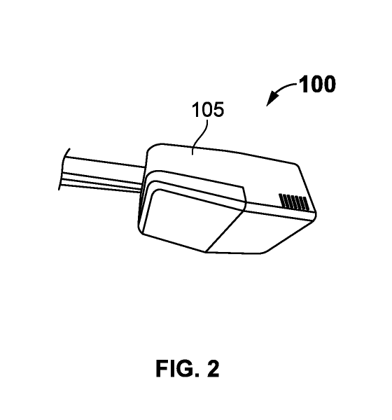

FIG. 2 illustrates a perspective view of the home safety device 100 in an embodiment of the present invention. The device 100 is conveniently mounted in the garage on a wall. The device 100 utilizes a wireless model such as Wi-Fi, Bluetooth to link with the garage door opener. In one embodiment, the device 100 utilizes a wired model for communication with the garage door opener. The home safety device 100 comprises a housing 105 encompassing all the electronic components of the device 100.

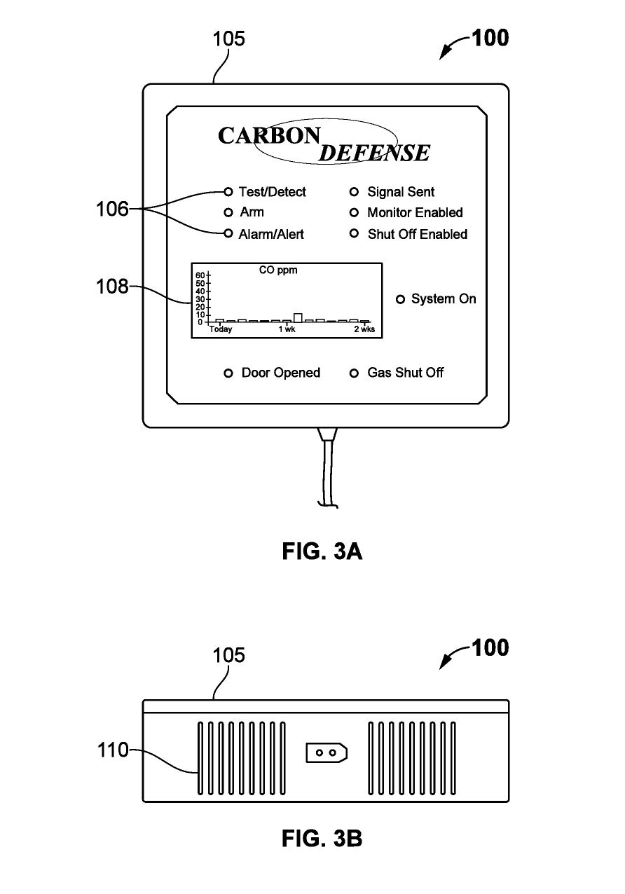

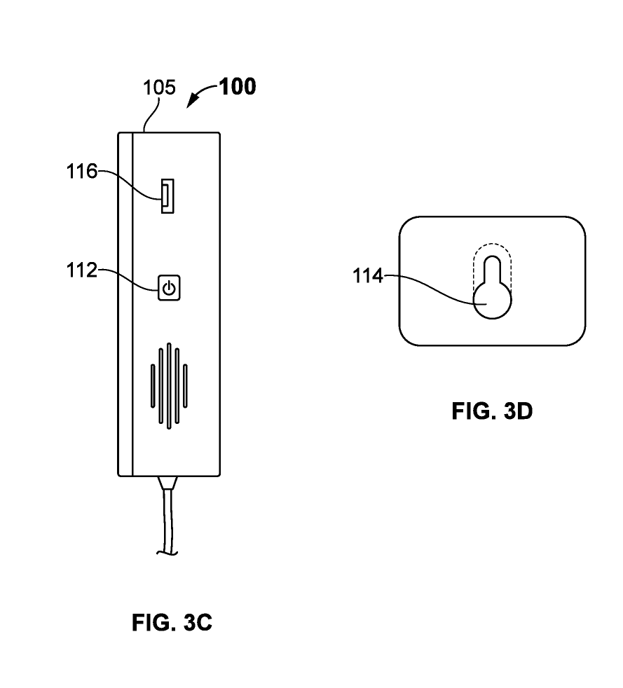

Referring to FIG. 3A, the device 100 further comprises a plurality of LED illuminators 106 at a front panel of the housing 105 to indicate the state and operation of the device 100. A transparent lens is disposed transparent over the LED illuminators 106 to prevent from inadvertent impacts. Further, a display 108 is disposed at the front panel of the housing 105. The display 108 is configured to display carbon monoxide history over past two weeks. In another embodiment, the device 100 comprises a battery backup which is powered with a 12 VDC adapter that is plugged into the house or office 120 VAC mains.

Referring to FIG. 3B, the home safety device 100 comprises slots 110 at a top and bottom side of the housing 105. The slots 110 are provided to allow free air flow to the replaceable CO detector module. The housing 105 further comprises screws at a rear side, which could be removed to access the carbon monoxide detection module. The device 100 further comprises at least two cable connectors. The device 100 could be connected to the garage door opener via the connectors. In one embodiment, the device 100 could be connected to the garage door opener via a standard RF, a remote link, a Wi-Fi link.

FIG. 3C illustrates a side view of the home safety device 100 in an embodiment of the present invention. The device 100 comprises a port 116 to connect with a computing device via a USB cable for software upgradation. In one embodiment, the software upgradation could be done using Bluetooth link or Wi-Fi. Referring to FIG. 3D, the housing 105 comprises a mounting keyhole 114 at a back panel of the housing 105. The device 100 is attached to the garage wall using a sheet rock expanding anchors and a pan head screws, which fits in the mounting keyhole 114 in the back panel. Further, the device 100 comprises a switch 112 to power on/off the device 100.

In one embodiment, the device 100 comprises the housing 105 and one or more printed circuit boards (PCB). In one embodiment, the housing 105 is a plastic housing. The housing 105 comprises two sections, which are injection molded from polycarbonate acrylonitrile butadiene styrene (ABS) plastic. The plastic material is durable, resistant to drop shocks, and resistant to household or automotive chemicals. The housings 105 could be coated with a vibrant color, so a distinctive color may be chosen to enhance the recognition factor of the device 100. The housing 105 is further configured to place polycarbonate LED lenses via adhesives. The housing 105 is held together using a plurality of stainless-steel machine screws. The mounting keyholes 114 are molded into the back section. Further, the device 100 is supplied with drywall anchors and pan head fasteners. In one embodiment, a logoized label is printed on a metalized plastic films with an adhesive on its back. The logo or text identifying the LEDs is printed on the shiny surface. The label is machine placed after the protective back is stripped off and the front section of the system is placed in the jig underneath the label.

In one embodiment, the printed circuit board (PCB) for the device 100 fabricated in accordance to the final assembler's requirements. Standard thickness, double sided FR4 circuit board material is populated with surface mounted components. Any through-hole devices are inserted after the surface mounted assembly, soldering, and cleaning. The circuit board is designed to have all the components oriented so they could be mounted with the plurality of LED illuminators 106 projecting out of the lenses mounted in the housings 105. After this assembly, PCBs are protected with a moisture adsorption preventive conformal coating. The device 100 further comprises one or more electronic components, but not limited to controlling computer or a controller, the display 108, a lighting device, the switch 112, one or more communication modules, a sound emitting device, and the carbon monoxide (CO) detection module, are included in the standard device without the gas line shutoff capability. The controlling computer is a dual core, 1.3 GHz ARM based computer chipset. This could source from a generation or two back cellular phone inventories to reduce programming and device cost. Memory of 16 GB or more and RAM 1 GB or more is sufficient.

In one embodiment, the display 108 is a touch screen display, which is a LED backlit IPS LCD capacitive touch screen with full color capability. The screen is 4.0″ diagonal at 326 pixels per inch. It is protected with Corning Gorilla Glass™ with oleophobic coating. In one embodiment, the lighting device is one or more LEDs. Low current LED devices are lighted using a remote LED driver controlled by the computer. Further, the LED indicate status and current conditions of the device 100 to aid in quickly determining the condition of the system. The lighting device further work in conjunction with the touch screen display. In one embodiment, the switch 112 is on/off switch, which is a sealed tactile membrane switch.

In some embodiments, the communication module is a wired or wireless communication module. In some embodiments, the communication module comprises a WiFi link, a Bluetooth link, or a RF link. In some embodiments, WiFi link is 802.11 a/b/g/n, dual band chip set. In some embodiments, the Bluetooth link is a 5.0, A2DP, and LE (long range) capable chip set. The Bluetooth link could send and receive up to 300′ in free air. In some embodiments, the RF link is a garage door RF link, where its transmitter operates on the security or two or more garage door opener standards at 310, 315, and 390 MHz. In some embodiments, the sound emitting device comprises an audio alert. The sound emitting device comprises a small piezoelectric sound emitter, which provides a low priority alarm at 2.0 KHz at 85 dBa. In some embodiments, the audio alert is programmed to emit two medium length pulses repeating every 5 seconds to conserve energy and draw attention.

In one embodiment, the device 100 is powered by using 120 VAC cord, and plug and smart charger. Section of the PCB comprises a power supply and a smart charger chip. It consumes 12 VDC power derived from a building 120 VAC mains, converts it to the two levels of DC used within the system. One drives the audible alarm and the other is used to charge the Li-Ion battery. The smart charger monitors the battery temperature, where it does not charge the battery, if it is elevated in temperature. The adapter has a 6′ cord to allow convenient connection to the device 100.

In some embodiments, the device 100 employs Li-ion battery, which is a high current density battery. The battery is packaged to fit in the housing 105. When fully charged, the battery could support normal operation over approximately 24 hours after loss of external power. In some embodiments, the CO detection module is designed with a connector on the PCB, and plug in base using one or more spade connectors. The detection modules further employ metal oxide semiconductor sensors. In one embodiment, the sensors detect changes in the conductivity of the chip as it contacts with the metal oxide surface.

In one embodiment, the device 100 could be communicated to the user device via a dedicated mobile application program. The application program is configured to allow the renter/homeowner to set up the system and determine the contact numbers and pre-programmed response for the device 100 upon notification of an alert condition. The user device could be any smartphone, tablet, or laptop computer with Wi-Fi or Bluetooth capability.

In one embodiment, the wired model of safety device 100 plugs directly into power outlet and is hardwired into the garage door opener. In another embodiment, battery powered model of safety device 100 with low voltage wires leading directly to door opener. Wireless model links to the door opener via transmitter/receiver. The device 100 could be installed with a remote unit on gas valve to shut off gas. The device 100 is further capable of connecting to any CO source such as gas stoves and heaters. The device 100 is configured to with any electric or battery back-up garage door opener. The device 100 could be monitored and activated using a dedicated mobile application. The device 100 automatically opens garage door when activated, or contacts user and emergency services. The device 100 further prevents tragic accidents or suicide attempt by a person.

Preferred embodiments of this invention are described herein, including the best mode known to the inventors for carrying out the invention. It should be understood that the illustrated embodiments are exemplary only, and should not be taken as limiting the scope of the invention.

The foregoing descriptions comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions. Although specific terms may be employed herein, they are used only in generic and descriptive sense and not for purposes of limitation. Accordingly, the present invention is not limited to the specific embodiments illustrated herein.

What is claimed is:

1. A home safety device disposed in an indoor environment, comprising:
a carbon monoxide detection module configured to monitor the level of carbon dioxide within the environment,
wherein the device is in communication with a garage door opener configured to generate an alert when carbon monoxide reaches a predetermined level in the environment and transmits a signal to the garage door opener to open the garage door.

2. The home safety device of claim 1, wherein the detection module comprises a metal oxide semiconductor sensor.

3. The home safety device of claim 1, further comprises a plurality of LED illuminators at a front panel of the housing configured to indicate a state and operation of the device.

4. The home safety device of claim 1, further comprises a transparent lens disposed over the LED illuminators to prevent from inadvertent impacts.

5. The home safety device of claim 1, further comprises a display configured to display carbon monoxide history of a predetermined time.

6. The home safety device of claim 1, further comprises one or more electronic components for operation of the device including a controller, a lighting device, a switch, one or more communication modules, and a sound emitting device.

7. The home safety device of claim 1, further comprises a casing enclosing the carbon monoxide detection module and one or more electronic components of the device.

8. The home safety device of claim 7, wherein the casing comprises one or more slots at a top and bottom side of the casing to allow free air to flow to the carbon monoxide detection module

9. The home safety device of claim 1, wherein the environments is a garage environment.

10. The home safety device of claim 1, wherein the carbon monoxide detection module is a replaceable carbon monoxide detection module.

11. The home safety device of claim 1, wherein the device is in communication with the garage door opener via at least two cable connectors.

12. The home safety device of claim 1, wherein the device is in communication with the garage door opener via at least one of a standard RF, a remote link, or a Wi-Fi link.

13. The home safety device of claim 5, wherein the display is a touch screen display.

14. The home safety device of claim 6, wherein the switch is a sealed tactile membrane switch.

15. The home safety device of claim 1, wherein the alert is an audio alert emitted from the sound emitting device comprising a piezoelectric sound emitter.

16. The home safety device of claim 1, wherein the device is in communication with a user device via a mobile application program to allow a user to control the device.

17. The home safety device of claim 1, is a battery powered device.

18. The home safety device of claim 1, further comprises drywall anchors and pan head fastener for mounting the device to the garage.
